(19) United States
(12) Patent Application Publication
(10) Pub. No.: US 2018/0250259 A1
(43) Pub. Date: Sep. 6, 2018

(54) DENTAL COMPOSITION FOR APPLYING A PREDETERMINED DOSE OF AN AGENT TO TEETH

(71) Applicant: Centrix, Inc., Shelton, CT (US)

(72) Inventor: Penggaung YU, Branford, CT (US)

(21) Appl. No.: 15/974,521

(22) Filed: May 8, 2018

Related U.S. Application Data
(63) Continuation of application No. PCT/US2016/061409, filed on Nov. 10, 2016.
(60) Provisional application No. 62/254,650, filed on Nov. 12, 2015.

(51) Int. Cl. A61K 31/327; A61K 33/16; A61K 47/36; A61K 47/44; A61K 9/107; A61P 1/02; A61Q 11/00; A61K 8/21

(52) U.S. Cl. CPC A61K 31/327; A61K 33/16; A61K 47/36; A61K 8/21; A61K 9/107; A61P 1/02; A61Q 11/00; A61K 47/44

(57) ABSTRACT

A therapeutic material delivered to a tooth surface in a predetermined dose. The therapeutic material is contained in an emulsion which is contained within a carrier. The carrier maintains contact with the tooth surface permitting the emulsion to deliver the therapeutic material to the tooth surface over a period of time. The therapeutic material may be a tooth whitening agent, such as carbamide peroxide, or a cavity or caries preventing agent, such as sodium fluoride.

### RELATED APPLICATIONS

This application is a continuation of International Application No. PCT/US2016/061409, with an international filing date of Nov. 10, 2016, which claims the benefit of U.S. Provisional Application No. 62/254,650 filed Nov. 12, 2015, all of which are herein incorporated by reference.

### FIELD OF THE INVENTION

The present invention relates in general to applying a predetermined dose of an agent or material to a surface and in particular to applying a whitening agent or fluoride treatment to the surface of a tooth.

### BACKGROUND OF THE INVENTION

It is often desirable to apply a material of a predetermined dose or concentration to a surface. One such been used to whitened teeth. When carbamide peroxide is dissolved in water free hydrogen peroxide is produced when temperature is increased or molecules of carbamide peroxide contact a surface. However, in high concentrations carbamide peroxide may cause burns to soft tissue such as skin and gums.

Therefore there have been many different approaches to whitened teeth in efforts to provide a whitening agent, such as carbamide peroxide, and a safe and effective way. In many applications a fitted rubber dam containing a bleaching agent such as hydrogen peroxide is placed in the mouth and over the teeth. This procedure is sometimes painful and time-consuming as well as inconvenient. Therefore there have been many efforts to make tooth whitening both easier and safer. One such effort is disclosed in U.S. Pat. No. 5,989,569 entitled “Delivery System for a Tooth Whitener Using a Permanently Deformable Strip of Material” issuing to Dirksing et al. on Nov. 23, 1999, which is herein incorporated by reference. Therein disclosed is a strip of material for delivering a tooth whitening substance applied to the strip of material. The strip of material has a yield point and thickness such that the strip of material substantially conforms to a shape of a tooth via permanent deformation under pressure of less than about 250,000 Pascals.

Another tooth whitening product is disclosed in U.S. Pat. No. 6,949,240 entitled “Tooth Whitening Product” issuing to Sagel et al. on Sep. 27, 2005, which is herein incorporated by reference. Therein disclosed is a tooth whitening product comprising a strip of material sized to cover the front surface of one or more teeth and soft tissue adjacent the front surface of the teeth. A tooth whitening composition containing a peroxide active with a concentration greater than about 7.5% is disposed on the strip of material.

While these prior devices have provided lower concentrations of tooth whitening agents and in particular hydrogen peroxide so as to be safer, they still require a structural piece or flexible strip to hold the whitening agent or other material. This is often uncomfortable and inconvenient for the user. Additionally, these trips have the potential to create a choking hazard.

It is also often desirable to apply other materials to a tooth surface in predetermined doses. For example, fluoride treatments are often applied to the surface of the tooth to prevent tooth decay. It is desirable to provide the fluoride at a predetermined safe dose. Direct application of solutions containing fluoride may result in concentrations of fluoride that are undesirable or even posing a health risk.

Therefore there is a need for a more practical and easier device and method to apply a material to the surface of a tooth in a predetermined concentration or dose.

### SUMMARY OF THE INVENTION

The present invention is an easy to apply material for the placement of a predetermined concentration of a therapeutic substance to a surface. An emulsion containing the therapeutic substance is dispersed through a carrier for application to a surface to be treated. In a preferred embodiment, a tooth whitening agent, such as carbamide peroxide, is in an aqueous solution and interacts with oil forming an emulsion that is dispersed in a carrier, such as wax. When the carrier containing the emulsion is applied to a tooth surface, the carbamide peroxide of a predetermined concentration is gradually applied to the tooth surface when emulsion droplets are gradually broken up. In another embodiment the therapeutic substance is a fluoride containing compound.

It is an object of the present invention to provide a predetermined concentration or dose of a therapeutic material to a surface to be treated.

It is an object of the present invention to provide a composition with a low total dose of a therapeutic agent delivered with a therapeutically effective concentration.

It is an advantage of the present invention that it is easy to apply and is safe and effective.

It is an advantage of the present invention that predetermined doses of a therapeutic agent can be safely applied.

It is yet another advantage of the present invention that predetermined doses of a therapeutic agent are released during a predetermined period of time over a tooth surface.

It is a feature of the present invention that an emulsion is formed containing a therapeutic agent that is dispersed in a carrier.

It is a feature of the present invention that the therapeutic agent may be contained in solution or precipitate form in the emulsion.

It is a feature of an embodiment of the present invention that different emulsions are dispersed within the carrier that when combined result in a desired therapeutic agent.

It is a feature of an embodiment of the present invention that a sodium fluoride emulsion and a potassium nitrate emulsion may combine to form potassium fluoride.

These and other objects, advantages, and features will become more readily apparent in view of the following more detailed description.

### BRIEF DESCRIPTION OF THE DRAWINGS

FIG. 1 schematically illustrates the present invention applied to a tooth surface.

FIG. 2A is an enlarged view of rectangle 2A illustrated in FIG. 1.

FIG. 2B schematically illustrates the emulsion contained and dispersed within the carrier.

FIG. 3 schematically illustrates the application of the emulsion containing a therapeutic agent onto a tooth surface.

FIG. 4 is an enlarged view schematically illustrating the application of the present invention to the whitening of teeth by removing stains.

FIG. 5 schematically illustrates an embodiment of the present invention in which two different emulsions are combined forming a desired reaction or therapeutic agent.

### DETAILED DESCRIPTION OF THE INVENTION

The present invention uses a carrier to apply an emulsion dispersed within the carrier. The emulsion contains a therapeutic agent. The therapeutic agent is preferably a tooth whitening material, such as carbamide peroxide. The therapeutic agent may also be a fluoride containing compound. The carrier may be a thickening agent, such as was, pullulan, guar gum or sodium polyacrylate.

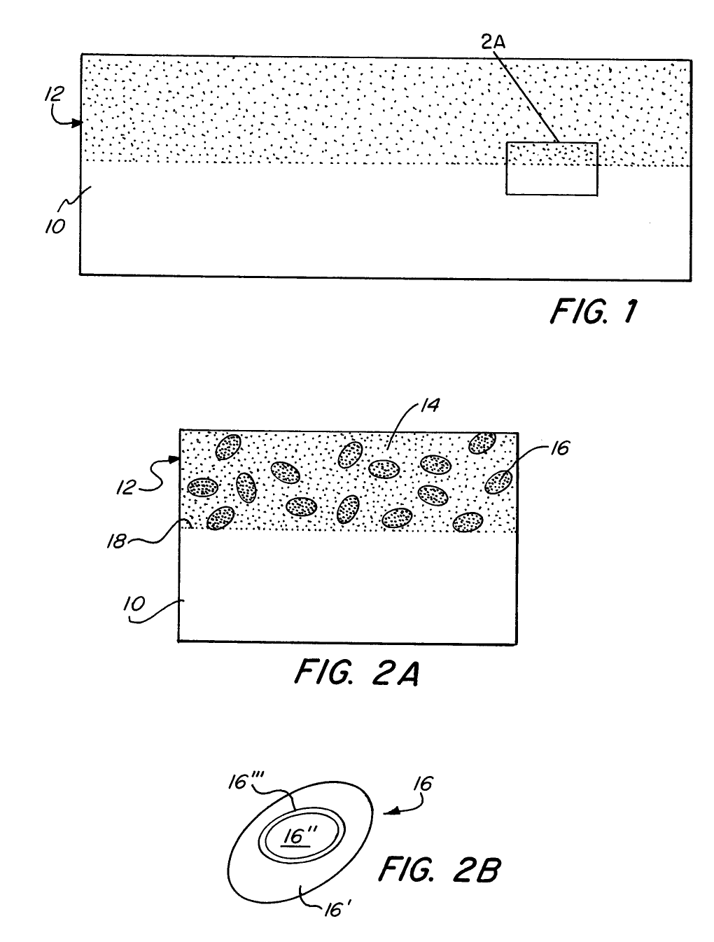

FIG. 1 schematically illustrates the application of the present invention to a tooth surface. A mixture 12, comprised of a carrier and an emulsion or colloid, is illustrated applied to a surface of a tooth 10. The mixture 12 of the emulsion containing the therapeutic agent dispersed within the carrier is placed in contact with the surface of the tooth 10. The mixture 12 may be applied by rubbing, brushing, or any other means. The therapeutic agent acts on the tooth surface and is retained and protected from removal by the carrier.

FIG. 2A is an enlarged view of rectangle 2A illustrated in FIG. 1. A droplet or particle 16 is dispersed within the carrier 14 of the mixture 12. The droplet or particle 16 comprises an emulsion containing a therapeutic agent. The droplet or particle 16 upon contact with a tooth surface 18 dispenses the predetermined dose of the therapeutic agent contained within the droplet or particle 16. The carrier 14 is preferably an inert malleable material, such as a wax, putty, paste, hard cream, or other similar material that permits easy spreading on the tooth surface 18 and is viscosity so as to be maintained on the tooth surface 18 for a desired period of time. The carrier 14 may be of a material that has an affinity or attraction to the tooth surface. The carrier 14 may also have the property of adhering to the tooth surface 18. The adherence or bonding may be relatively weak permitting easy removal or wearing off in a predetermined time. The carrier 14 can hold the droplet or particle 16 in contact with the surface 18 of the tooth 10 so as to provide time for the therapeutic agent contained within the droplet or particle 16 to act on the surface 18 of the tooth 10. In a preferred embodiment the therapeutic agent is a tooth whitening agent. The carrier may also be an edible substance such as a confectionery, pectin, pullulan, or other eatable polymer that when chewed dispenses the emulsion and therapeutic agent contained within the droplet or particle 16 onto the tooth surface 18.

FIG. 2B is an enlarged view schematically illustrating the droplet or particle 16 illustrated in FIG. 2A. The droplet or particle 16 is an emulsion comprising a continuous phase 16′, which may be oil, a dispersed phase 16″, which may be an aqueous solution with a therapeutic agent, and a surfactant layer or membrane 16″′. The membrane 16″′ comprises a monolayer of surfactant molecules surrounding the water phase in the emulsion mix.

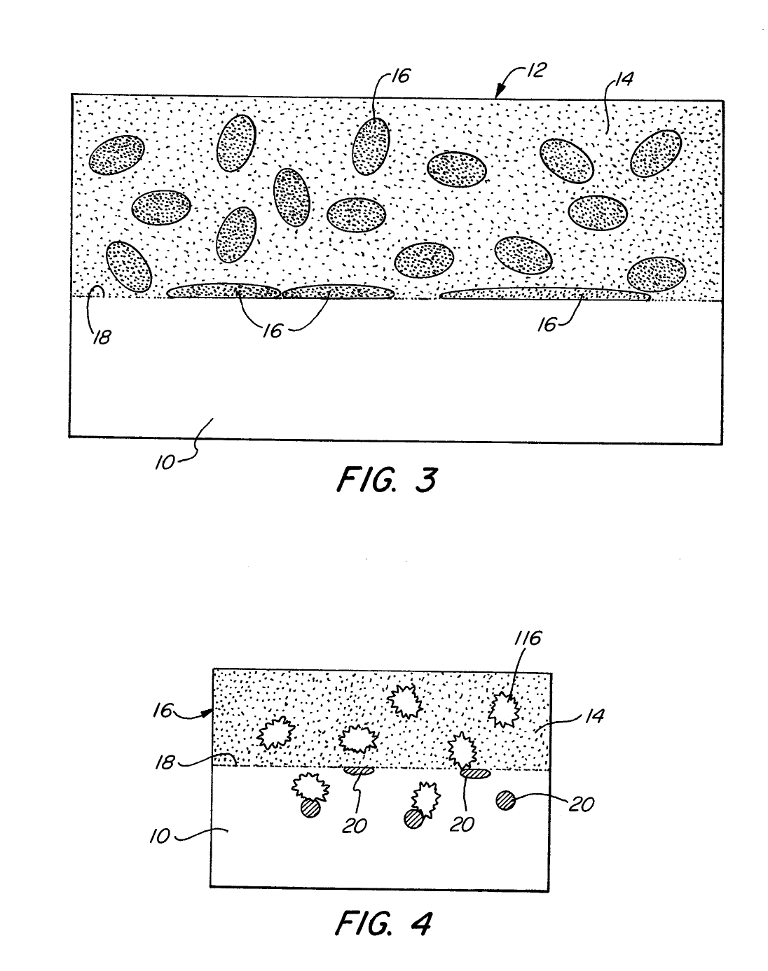

FIG. 3 schematically illustrates the placement of the particle 16 dispersed within the carrier 14 in mixture 12 on a tooth surface 18 of a tooth 10. The droplets or particles 16 contact the tooth surface 18 upon the spreading of the mixture 12 onto the tooth surface 18. A number or plurality of droplets or particles 16 spread over the tooth surface 18 placing the therapeutic agent in the emulsion onto the tooth surface 18.

FIG. 4 is an enlarged view illustrating the application of the present invention to a tooth whitening therapeutic substance when droplets or particles 16 contact the tooth surface 18. During application of the tooth whitening therapeutic substance the droplets or particles 16 burst over the tooth surface 18. Reagents 116 contained in the droplets or particles 16 comprising the continuous phase 16′ and the dispersed phase 16″, illustrated in FIG. 2B, are in contact with a tooth surface 18 of a tooth 10 containing stains 20. The reagents 116 contained in an emulsion in the form of an aqueous solution can be carbamide peroxide in a predetermined strength, for example, by weight, of preferably between 5% and 40%, and more preferably between 6% and 25%.

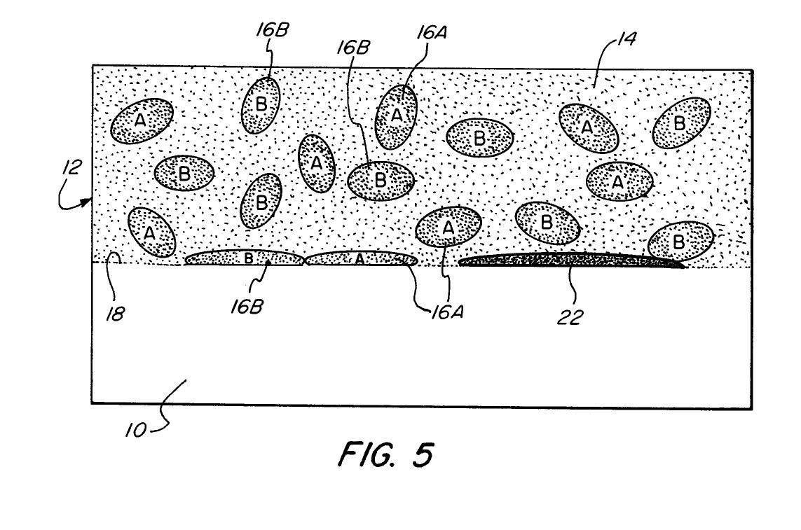

FIG. 5 schematically illustrates another embodiment of the present invention. In this embodiment multiple or a plurality of different components are contained in emulsions contained within different droplets or particles so that when combined on a surface form a desired reaction or therapeutic agent. As is schematically illustrated, droplets or particles 16A contain an emulsion comprising component A and droplets or particles 16B contained an emulsion comprising component B. When the two different droplets or particles 16A and 16B combined forming a combined droplet or particle 22, the components A and B are combined causing a reaction or desired agent to act upon the tooth surface 18 of the tooth 10.

A composition incorporating the present invention is illustrated in Example 1 below.

### EXAMPLE 1

Emulsified wax with papain and amorphous calcium phosphate.

TABLE 1
Table of Contents

| Component | Weight (grams) | Percent by weight |
|---|---|---|
| Distilled water | 40.0 | 15.53 |
| Papain | 5.0 | 1.94 |
| Amorphous Calcium | 2.5 | 0.98 |
| Phosphate | | |
| Polysorbate 60 | 5.0 | 1.94 |
| Microcrystalline wax | 205.0 | 79.61 |
| 170/180 | | |
| Total | 257.5 | 100 |

The composition in Example 1 was made by the following process.

### The Water Phase

In one glass container the distilled water was heated to approximately 50° C. The papain was added while stirring continuously until dissolved. The amorphous calcium phosphate was then added while continuing to stir until dispersed completely.
In another glass container the polysorbate 60 was warmed on a hot plate. The papain and amorphous calcium phosphate was poured into the warmed polysorbate 60 and slowly stirred until dissolved, while avoiding producing bubbles.

### The Wax Phase

In a stainless steel container microcrystalline wax 170/180 was heated to approximately 90° C. and raised to approximately 104° C. briefly to ensure complete melting of the microcrystalline wax 170/180.

### Mixing

While the microcrystalline wax 170/180 is melted, remove from heat and slowly stir the microcrystalline wax 170/184 adding the warmed liquid from the water phase. Stirring should be continued until the microcrystalline wax 170/180 cools down completely.

Another composition incorporating the present invention is illustrated in Example 2 below, including Samples 1-4.

### EXAMPLE 2

Carbamide Peroxide and Oil Emulsion

### Example 2, Sample 1

Emulsified Ten Percent Carbamide Peroxide

TABLE 1A
Contents of emulsion mix

| | Desired | Desired | Real weight |
|---|---|---|---|
| Items | Percentage | weight | (Grams) |
| Solution of carbamide peroxide | 30.00% | 150.00 | 150.19 |
| Polysorbate 60 | 10.00% | 50.00 | 50.00 |
| Vitamin E Oil | 60.00% | 300.00 | 303.27 |

TABLE 1B
Solution of Carbamide Peroxide

| | Desired | Desired | Real weight |
|---|---|---|---|
| Items | Percentage | weight | (Grams) |
| Carbamide Peroxide in water | 10.00% | 15.00 | 15.02 |
| Water | 90.00% | 135.00 | 135.17 |

TABLE 1C
Total ingredients

| | Real weight | Real |
|---|---|---|
| Items | (Grams) | percentage |
| Polysorbate 60 | 50.00 | 9.93% |
| Vitamin E Oil | 303.27 | 60.24% |
| Carbamide Peroxide in water | 15.02 | 2.98% |
| Water | 135.17 | 26.85% |
| Total | 503.46 | 100.00% |

### Example 2, Sample 2

Emulsified Fifteen Percent Carbamide Peroxide

TABLE 2A
Contents of emulsion mix

| | Desired | Desired | Real weight |
|---|---|---|---|
| Items | Percentage | weight | (Grams) |
| Solution of carbamide peroxide | 30.00% | 150.00 | 149.65 |
| Polysorbate 60 | 10.00% | 50.00 | 50.00 |
| Vitamin E Oil | 60.00% | 300.00 | 299.47 |

TABLE 2B
Solution of Carbamide Peroxide

| | Desired | Desired | Real weight |
|---|---|---|---|
| Items | Percentage | weight | (Grams) |
| Carbamide Peroxide in water | 15.00% | 22.50 | 22.51 |
| Water | 85.00% | 127.50 | 127.14 |

TABLE 1C
Total ingredients

| | Real weight | Real |
|---|---|---|
| Items | (Grams) | percentage |
| Polysorbate 60 | 50.00 | 10.02% |
| Vitamin E Oil | 299.47 | 60.00% |
| Carbamide Peroxide in water | 22.51 | 4.51% |
| Water | 127.14 | 25.47% |
| Total | 499.12 | 100.00% |

### Example 2, Sample 3

Emulsified Twenty Percent Carbamide Peroxide

TABLE 3A
Contents of emulsion mix

| | Desired | Desired | Real weight |
|---|---|---|---|
| Items | Percentage | weight | (Grams) |
| Solution of carbamide peroxide | 30.00% | 150.00 | 150.33 |
| Polysorbate 60 | 10.00% | 50.00 | 50.00 |
| Vitamin E Oil | 60.00% | 300.00 | 299.97 |

TABLE 3B
Solution of Carbamide Peroxide

| | Desired | Desired | Real weight |
|---|---|---|---|
| Items | Percentage | weight | (Grams) |
| Carbamide Peroxide in water | 20.00% | 30.00 | 30.02 |
| Water | 80.00% | 120.00 | 120.31 |

TABLE 3C
Total ingredients

| | Real weight | Real |
|---|---|---|
| Items | (Grams) | percentage |
| Polysorbate 60 | 50.00 | 9.99% |
| Vitamin E Oil | 299.97 | 59.96% |
| Carbamide Peroxide in water | 30.02 | 6.00% |
| Water | 120.31 | 24.05% |
| Total | 500.30 | 100.00% |

### Example 2, Sample 4

Emulsified Six Percent Carbamide Peroxide

TABLE 4A
Contents of emulsion mix

| | Desired | Desired | Real weight |
|---|---|---|---|
| Items | Percentage | weight | (Grams) |
| Solution of carbamide peroxide | 30.00% | 75.00 | 74.99 |
| Polysorbate 60 | 10.00% | 25.00 | 25.02 |
| Vitamin E Oil | 60.00% | 150.00 | 150.32 |

TABLE 4B
Solution of Carbamide Peroxide

| | Desired | Desired | Real weight |
|---|---|---|---|
| Items | Percentage | weight | (Grams) |
| Carbamide Peroxide in water | 6.00% | 4.50 | 4.50 |
| Water | 94.00% | 70.50 | 70.49 |

TABLE 4C
Total ingredients

| | Real weight | Real |
|---|---|---|
| Items | (Grams) | percentage |
| Polysorbate 60 | 25.02 | 9.99% |
| Vitamin E Oil | 150.32 | 60.05% |
| Carbamide Peroxide in water | 4.50 | 1.80% |
| Water | 70.49 | 28.16% |
| Total | 250.33 | 100.00% |

The composition in Samples 1-4 listed in Example 2 were made by the following process.

Emulsion with Carbamide Peroxide and Vitamin E oil.

### Making Water Phase

In a glass container add polysorbate 60, water, and carbamide peroxide. The polysorbate 60 will dissolve quicker if added to hot water before adding the carbamide peroxide. This may save processing time.

### Making Oil Phase

In another glass container, heat vitamin E oil and polysorbate 60 to 60° C. while stirring them let it cool down to room temperature.
Amount of polysorbate 60 can be split. Equal parts of polysorbate 60 may be used in the water phase and the oil phase.

### Mixing

Mix the water phase and the oil phase at room temperature by pouring the water phase into the oil phase while stirring.

This carbamide peroxide and vitamin E emulsion may be mixed with a carrier such as microcrystalline wax, or even a confectionery for disturbing the emulsion onto the surface of the tooth. The confectionery may be chewable so that when chewed the emulsion will be distributed onto the surface of the tooth.

In the formulations of the present invention the polysorbate 60 acts as an emulsifier. The carbamide peroxide, also known as hydrogen peroxide-urea, is a whitening agent and is capable of whitening teeth. Papain is an enzyme present in papaya. Papain has a tooth whitening effect. The vitamin E oil has a desired viscosity and acts as an emulsion stabilizer, is soothing to tissue, and prevents a burning feeling. However, other oils may be used such as mineral oil, vegetable oil, fruit oil, or other known substantially equivalent oils.

The formulations of the present invention have high water content and an oil base with the water phase carrying carbamide peroxide which releases hydrogen peroxide for whitening teeth. The high water content in the emulsion is to ensure that sufficient amounts of carbamide peroxide are carried in the water to safely and effectively whiten teeth.

Following is a preferred formulation of a tooth whitening composition including a microcrystalline wax carrier.

### EXAMPLE 3

TABLE 5
Total Ingredients

| | Weight | |
|---|---|---|
| Contents | (grams) | Percentage |
| Drakeol 35 Mineral Oil USP | 156.94 | 38.14% |
| Ozokerite Wax 2095 | 167.44 | 40.69% |
| Vitamin E Oil (Total in two | 59.11 | 14.36% |
| phases) | | |
| Amorphous Calcium Phosphate | 6.77 | 1.65% |
| (ACP) | | |
| Distilled water media with | 9.11 | 2.21% |
| carbamide Peroxide | | |
| Polusorbate 60 | 3.04 | 0.74% |
| Carbamide Peroxide in water | 0.55 | 0.13% |
| Water | 8.57 | 2.08% |

In the above Example 3 it should be noted that the concentration of carbamide peroxide in the final product is 0.14% by weight, but the actual concentration in aqueous solution within the emulsion is approximately 6%. Therefore, when the emulsified droplet of aqueous solution breaks up at the surface of the tooth, a 6% carbamide peroxide solution is released to whiten the teeth. This process continues until all the wax is dissipated or removed. Therefore, carbamide peroxide in the mouth is minimal but the carbamide peroxide in the desired percentage concentration of 6% is effectively and safely delivered to the surface of the teeth for whitening. The present invention provides a safe and effective means for delivering a predetermined concentration of a whitening agent to the surface of the teeth and minimizes the potentially harmful effects of high concentrations of carbamide peroxide. The concentration of carbamide peroxide in the emulsion can range from 5% to 45%.

The present invention may be applied in another embodiment to administer a fluoride containing compound as the therapeutic agent to a tooth. Such compositions are illustrated in the following Examples 4-6.

### EXAMPLE 4

### Tables 6A-C

TABLE 6A
Contents of emulsion mix

| Items | Desired Percentage | Desired weight (g) |
|---|---|---|
| Solution of Sodium Fluoride | 30.00% | 150.00 |
| Polysorbate 60 | 10.00% | 50.00 |
| Vitamin E Oil | 60.00% | 300.00 |

TABLE 6B
Solution of Sodium Fluoride

| Items | Desired Percentage | Desired weight (g) |
|---|---|---|
| Sodium Fluoride | 5.00% | 7.50 |
| Water | 95.00% | 142.50 |

TABLE 6C
Total ingredients

| Items | Desired weight (g) | Final percentage |
|---|---|---|
| Polysorbate 60 | 50.00 | 10.00% |
| Vitamin E Oil | 300.00 | 60.00% |
| Sodium Fluoride | 7.50 | 1.50% |
| Water | 142.50 | 28.50% |
| Total | 500.00 | 100.00% |

In preparing the composition illustrated in Example 4, in Table 6B the solution of sodium fluoride is prepared at a 5% concentration but may be prepared with a concentration range of 0.5% to 5%. In preparing the solution of sodium fluoride with greater than approximate 3.5% concentration it is necessary to elevate or increase the water temperature for the sodium fluoride to dissolve or go into solution. When a greater than 3.5% concentration of sodium chloride solution is prepared and contained within the emulsion mix in Table 6A, upon cooling from the elevated temperature sodium fluoride crystals will precipitate in the emulsion mix resulting in a combination of a quantity of sodium fluoride in solution as well as a quantity of sodium fluoride as a precipitate in crystalline form. If cooled faster than the rate of precipitation, the solution will become supersaturated until the solute precipitates to the temperature determined saturation point. The crystals of sodium fluoride will be much smaller in size than possible by conventional milling or grinding of sodium fluoride. The crystal size may be less than a micron or 0.001 mm. The small size of the precipitated sodium fluoride enhances the therapeutic effect when applied to the tooth. Accordingly, in this embodiment the present invention provides improved delivery of sodium fluoride than previously possible. Different ratios of solution or water and vitamin E oil may be used to provide different consistencies or viscosity of the composition as desired.

More specifically, the solution is prepared at the boiling temperature, or 100° C. or 212° F. At this temperature, the solubility of sodium fluoride is about 5%. The emulsion mix is prepared at this temperature so that the sodium fluoride remains soluble.

After formation of the emulsion mix, the emulsion mix is cooled down naturally to room temperature, such as approximately 22° C. or 72° F. At this temperature, the solubility of sodium fluoride is about 4%. There is about a 1% of the sodium fluoride that will precipitate out of solution to form tiny crystal particles that remain inside an emulsion droplet.

In general, size of the emulsion droplet is at mean value of about 10 microns, its size is also dependent on energy being introduced into the solution during the emulsion preparation process, this energy may come from either stirring, ultrasound, or both combined. This size of the emulsion droplet determines the size limit of the crystal particle of sodium fluoride, which is much less than the size of the emulsion droplet itself.

For this reason, the sodium fluoride particles are relatively small and in the range of submicron to microns. This size range is usually not achievable by grinding, sieving or milling.

When the emulsion droplet burst over a teeth surface, a small solid sodium fluoride crystal or particle is instantly released from the emulsion droplet and thereafter the sodium fluoride particle is dissolved quickly by the surrounding saliva from the patient. The small size of the solid sodium fluoride crystal or particle delivers the benefit of quick release of sodium fluoride onto teeth at a higher concentration, in comparison of regular or prior mechanically ground sodium fluoride particles, which are in a range of 50-125 microns, even though this size of the mechanically ground particles are considered to be fine powder, these mechanically ground particles cannot be dissolved quickly enough by saliva inside a mouth and therefore end up being swallowed and entering into the patient's digestion system.

Table 6A, 6B and 6C illustrate the composition of the emulsion mix. Wax carrier is not included. However, the emulsion mix may be mixed with a wax carrier for dispensing the emulsion mix on a patient's tooth.

### EXAMPLE 5

### Tables 7A-C

TABLE 7A
Contents of emulsion mix

| Items | Desired Percentage | Desired weight (g) |
|---|---|---|
| Solution of Sodium Fluoride | 30.00% | 150.00 |
| Polysorbate 60 | 10.00% | 50.00 |
| Vitamin E Oil | 60.00% | 300.00 |

TABLE 7B
Solution of Sodium Fluoride

| Items | Desired Percentage | Desired weight |
|---|---|---|
| Sodium Fluoride | 2.50% | 3.75 |
| Water | 97.50% | 146.25 |

TABLE 7C
Total ingredients

| Items | Real weight (g) | Real percentage |
|---|---|---|
| Polysorbate 60 | 50.00 | 10.00% |
| Vitamin E Oil | 300.00 | 60.00% |
| Sodium Fluoride | 3.75 | 0.75% |
| Water | 146.25 | 29.25% |
| Total | 500.00 | 100.00% |

The composition illustrated in Example 5, as indicated in Table 7B has a 2.50% concentration of sodium fluoride. This concentration of 2.50% of sodium fluoride will be maintained in solution at room temperature, approximately 20° C. or 68° F. Therefore, in this embodiment at room temperature no sodium fluoride precipitate will be formed.

This concentration of sodium fluoride is currently well accepted by the dental market. A saliva stimulant, such as ascorbic acid, can be added into the emulsion solution at a percentage of 1-5%. When a wax carrier is used, the breaking up of the wax carrier coating requires saliva and movement of teeth and lips. For some patients who have dry mouth syndrome, the saliva stimulant can be helpful to stimulate production of saliva, and thereafter, helps to deliver an effective fluoride treatment.

### EXAMPLE 6

### Tables 8A-G

TABLE 8A
Solution of Sodium Fluoride

| Items | Desired Percentage | Desired weight (g) |
|---|---|---|
| Sodium Fluoride | 5.00% | 7.50 |
| Water | 95.00% | 142.50 |

TABLE 8B
Solution of Potassium Nitrate

| Items | Desired Percentage | Desired weight (g) |
|---|---|---|
| Potassium Nitrate | 2.00% | 3.00 |
| Water | 98.00% | 147.00 |

TABLE 8C
Contents of emulsion mix-1

| Items | Desired Percentage | Desired weight (g) |
|---|---|---|
| Solution of Sodium Fluoride | 30.00% | 150.00 |
| Polysorbate 60 | 10.00% | 50.00 |
| Vitamin E Oil | 60.00% | 300.00 |

TABLE 8D
Contents of emulsion mix - 2

| Items | Desired Percentage | Desired weight (g) |
|---|---|---|
| Solution of potassium Nitrate | 30.00% | 150.00 |
| Polysorbate 60 | 10.00% | 50.00 |
| Vitamin E Oil | 60.00% | 300.00 |

TABLE 8E
Content of Wax Blend Carrier

| | | Desired |
|---|---|---|
| Contents | Desired weight (g) | Percentage |
| Drakeol 35 Mineral Oil USP | 269.52 | 33.69% |
| Ozok Wax 2095 | 320.00 | 40.00% |
| Vitamin E Oil (DSM) | 120.00 | 15.00% |
| MicroWax Blend Wax 599P | 80.00 | 10.00% |
| Pepermint Oil (Piperita Organic, | 0.00 | 0.00% |
| optional) | | |
| Amorphous Calcium Phosphate (ACP) | 10.48 | 1.31% |
| Total | 800.00 | |

TABLE 8F
Total ingredients

| Contents | Desired weight (g) | Percentage |
|---|---|---|
| Drakeol 35 Mineral Oil USP | 269.52 | 26.95% |
| Ozok Wax 2095 | 320.00 | 32.00% |
| Vitamin E Oil (DSM) | 120.00 | 12.00% |
| MicroWax Blend Wax 599P | 80.00 | 8.00% |
| Pepermint Oil (Piperita Organic, | 0.00 | 0.00% |
| optional) | | |
| Amorphous Calcium Phosphate (ACP) | 10.48 | 1.05% |
| Polysorbate 60 | 20.00 | 2.00% |
| Vitamin E Oil | 120.00 | 12.00% |
| Sodium Fluoride | 1.50 | 0.15% |
| Potassium Nitrate | 0.60 | 0.06% |
| Water | 57.90 | 5.79% |
| Total | 1000.00 | 100.00% |

The composition in Example 6 illustrates an embodiment of the present invention that utilizes components in two different emulsions that when combined form a desired therapeutic agent. As illustrated in Table 8A a solution of sodium fluoride is prepared and as illustrated in Table 8B a solution of potassium nitrate is prepared. The solution of sodium fluoride is used to make a first emulsion mix, as illustrated in Table 8C. The solution of potassium nitrate is used to make a second emulsion mix, as illustrated in Table 8D. A wax blend carrier is prepared as illustrated in Table 8E. The wax blend carrier is mixed with the first and second emulsion mixes. The first and second emulsion mixes may be of equal portions and the wax and emulsion mix may be mixed with a ratio of 4 to 1. However, this ratio may be adjusted to obtain the desired consistency or viscosity of the final composition. Table 8F illustrates the ingredients of the final composition.

In the embodiment of Example 6, when the sodium fluoride and the potassium nitrate combine upon application of the composition to a tooth the sodium fluoride and potassium nitrate will form potassium fluoride. In Example 6 the concentration of potassium fluoride delivered to the tooth will be approximately a 2% concentration. Potassium fluoride delivered in accordance with the present invention permits the potassium fluoride to be drawn into the tubules or porous structure of the tooth to more effectively deliver the fluoride compound to the tooth. Sensitivity of the tooth may also be reduced.

Potassium fluoride in high level or doses may be harmful. However, in Example 6 of the present invention the overall level or dose of potassium fluoride is relatively low yet the delivered concentration is therapeutically effective. In Example 6 the concentration of potassium fluoride delivered is approximately a 2% concentration, however, the overall percent of potassium fluoride in the composition is 0.06%, which is not harmful. The emulsion effectively targets the delivery of the therapeutic agent so that higher concentrations of the therapeutic agent may be delivered with overall safe low levels.

Specifically, the sample is composed of two types of emulsion mixes carrying different emulsion mixes or reagents for treatment. The reagents cannot normally be mixed in advance, or the reaction between two or more reagents can be either unstable, unsafe to handle, poisonous or harmful. However, some reagents can be more medically beneficial if a correct concentration or dose or a minimal dose is securely maintained.

Potassium nitrate has a function of de-polarizing nerve endings, causing them to respond slowly. Potassium nitrate is widely used as de-sensitizer. Sodium fluoride is used for treatment of re-mineralization. By mixing these two chemicals together, potassium fluoride can be formed in an equilibrium form as a temporary product of a reaction. Because ionic potassium can penetrate into tubules of the tooth deeply and bound to the nerve end, it will bring the ionic fluoride together with it, the resulting benefit is that fluoride penetrates deeper into the dentin of the tooth, and restores deeper cracks in the tooth enamel as well.

Potassium fluoride is a product of reaction between sodium fluoride and potassium nitrate. It exists in the solution at equilibrium level. In this Example 6, solution of 2% potassium nitrate is used, when two emulsion mixes are mixed, there is no chemical reaction that occurs because each of the emulsion droplets remain individually and are stable within the emulsion mixes.

Once the droplets of both of the two different emulsion mixes burst, each of the two reagents, such as sodium fluoride and potassium nitrate, will interact with teeth, at the same time, resulting in a reaction between these two reagents producing an equilibrium form of potassium fluoride, which will deeply penetrate into tubules and cracks in enamel of the tooth.

The Example 6 illustrates a 2% potassium nitrate solution, while the sodium fluoride solution is 5%, accordingly, a maximum of 2% potassium fluoride can be produced due to the reaction. However, with consideration of the emulsion mix, maximal of 0.3% of potassium fluoride can be produced, see table 8D. Furthermore, within the wax carrier, the final concentration of potassium nitrate is only 0.06%, the maximal potassium fluoride will be no more than 0.06%. This concentration is far below any toxic threshold.

Saliva stimulant can be added in either the sodium fluoride solution or potassium nitrate solution or both, this is particularly beneficial to dry mouth patients.

Therefore, the present invention makes possible the delivery of a therapeutically effective concentration of a therapeutic agent while keeping the overall total or dose of the therapeutic agent low.

In some applications ascorbic acid may be added to the composition. Ascorbic acid will stimulate the excretion of saliva, especially in older patients. This excretion of saliva will aid in the administering of the therapeutic agent.

While the present invention has been described with respect to several different embodiments, it will be obvious that various modifications may be made without departing from the spirit and scope of this invention.

What is claimed is:

1. A dental composition comprising:
a carrier;
a therapeutic agent held in an emulsion or colloid dispersed within the carrier,
whereby a predetermined dose of agent is capable of being delivered to a surface of a tooth.

2. A dental composition as in claim 1 wherein:
said carrier comprises a wax

3. A dental composition as in claim 1 wherein:
said carrier comprises a confectionary

4. A dental composition as in claim 1 wherein:
said therapeutic agent comprises a bleaching agent.

5. A dental composition as in claim 4 wherein:
said bleaching agent comprises carbamide peroxide.

6. A dental composition as in claim 1 wherein:
said carrier comprises a thickening agent.

7. A dental composition as in claim 6 wherein:
said thickening agent is selected from the group consisting of guar gum and sodium polyacrylate.

8. A dental composition as in claim 1 wherein:
said therapeutic agent comprises a fluoride compound.

9. A dental composition as in claim 8 wherein:
said fluoride compound comprises sodium fluoride.

10. A dental composition having a predetermined concentration of a therapeutic agent for treating a tooth surface comprising:
a carrier; and
an emulsion dispersed within said carrier, said emulsion comprising the therapeutic agent as a dispersed phase in a dispersion medium and a surfactant,
whereby the therapeutic agent is capable of being applied to the tooth surface over a period of time with a predetermined concentration.

11. A dental composition having a predetermined concentration of a therapeutic agent for treating a tooth surface as in claim 10 wherein:
a first quantity of the therapeutic agent is in solution and a second quantity of the therapeutic agent is a precipitate.

12. A dental composition having a predetermined concentration of a therapeutic agent for treating a tooth surface as in claim 11 wherein:
the therapeutic agent comprises a fluoride compound.

13. A dental composition having a predetermined concentration of a therapeutic agent for treating a tooth surface as in claim 12 wherein:
the fluoride compound comprises a concentration between 2.5% and 5.0%.

14. A dental composition having a predetermined concentration of a therapeutic agent for treating a tooth surface as in claim 10 wherein:
the therapeutic agent comprises a fluoride compound with a concentration less than 2.5%

15. A dental composition having a predetermined concentration of a therapeutic agent for treating a tooth surface comprising:
a carrier; and
an first emulsion dispersed within said carrier, said first emulsion comprising a first component as a dispersed phase in a dispersion medium and a surfactant;
a second emulsion dispersed within said carrier, said second emulsion comprising a second component as a dispersed phase in a dispersion medium and a surfactant;
wherein upon the first component mixing with the second component the therapeutic agent is formed,
whereby the therapeutic agent is capable of being applied to the tooth surface over a period of time with a predetermined concentration.

16. A dental composition having a predetermined concentration of a therapeutic agent for treating a tooth surface as in claim 15 wherein:
the first component comprises sodium fluoride, the second component comprises potassium nitrate, and the therapeutic agent comprise potassium fluoride.

17. A dental composition having a predetermined concentration of a therapeutic agent for treating a tooth surface comprising:
a carrier; and
an emulsion dispersed within said carrier, said emulsion comprising a first quantity of the therapeutic agent as a dispersed phase in a dispersion medium and a second quantity of the therapeutic agent as a precipitate,
whereby the therapeutic agent is capable of being applied to the tooth surface over a period of time with a predetermined concentration.

18. A method of making a dental composition comprising the steps of:
heating a solution comprising a therapeutic agent forming a saturated solution at a saturation temperature;
forming an emulsion mix comprising the saturated solution at the saturation temperature;
cooling the emulsion mix below the saturation temperature,
whereby a first portion of the therapeutic agent precipitates out of the solution and a second portion of the therapeutic agent remains in the solution.

19. A method of making a dental composition as in claim 18 wherein:
the therapeutic agent comprises a fluoride containing compound.
